(19) United States
(12) Patent Application Publication
(10) Pub. No.: US 2018/0344200 A1
(43) Pub. Date: Dec. 6, 2018

(54) ELECTRICAL IMPEDANCE TOMOGRAPHY USING THE INTERNAL THORACIC VEIN

(71) Applicant: CARDIAC PACEMAKERS, INC., St. Paul, MN (US)

(72) Inventors: PRAMODSINGH HIRASINGH THAKUR, WOODBURY, MN (US); JEFFREY E. STAHMANN, RAMSEY, MN (US); VIKTORIA A. AVERINA, SHOREVIEW, MN (US); QI AN, BLAINE, MN (US); G. SHANTANU REDDY, MINNEAPOLIS, MN (US); JAMES O. GILKERSON, STILLWATER, MN (US); KEITH R. MAILE, NEW BRIGHTON, MN (US)

(73) Assignee: CARDIAC PACEMAKERS, INC., St. Paul, MN (US)

(21) Appl. No.: 15/993,021

(22) Filed: May 30, 2018

Related U.S. Application Data
(60) Provisional application No. 62/513,062, filed on May 31, 2017.

(51) Int. Cl. A61B 5/053; A61B 5/00; A61B 5/08; A61B 5/091; A61B 5/027; A61B 5/029; A61B 5/0295

(52) U.S. Cl. CPC A61B 5/0536; A61B 5/0538; A61B 5/6876; A61B 5/0809; A61B 5/091; A61B 5/0816; A61B 2576/023; A61B 5/0044; A61B 5/029; A61B 5/0295; A61B 5/742; A61B 2562/0209; A61B 2562/043; A61B 5/027

(57) ABSTRACT

IMD devices, systems, and treatment methods are discussed and disclosed. An IMD having electrodes for placement in an internal thoracic vein (ITV) of a patient may be employed to facilitate electrical impedance tomography (EIT) imaging. An alternating current (A/C) may be applied using the electrodes and impedances may be determined that are useful to produce a tomographic image.

### CROSS REFERENCE TO RELATED APPLICATIONS

The present application claims the benefit of and priority to U.S. Provisional Patent Application Ser. No. 62/513,062, filed on May 31, 2017 and titled ELECTRICAL IMPEDANCE TOMOGRAPHY USING THE INTERNAL THORACIC VEIN, the disclosure of which is incorporated herein by reference.

### BACKGROUND

Electrical Impedance tomography (EIT) is an imaging technique that uses impedance measurements between multiple circumferential electrodes in a plane to separate tissue components based on differential impedance. EIT has been found to particularly useful for monitoring certain body parts and active research and development efforts are being made to broaden the applicability of EIT imaging. Moreover, recent advances in EIT technology as well as the lower number of electrodes required for recording parameters in individuals has led to increased interest in EIT imaging. With such interest, new and alternative methods of generating an EIT are desired, including for use in implantable devices.

### OVERVIEW

The present inventors have recognized that the internal thoracic veins (ITVs) and/or the intercostal veins and azygous veins may provide an opportunity for circumferential impedance mapping on the thoracic transverse plane in an implantable medical device. Such mapping may take the form of EIT or may be used to provide a surrogate for EIT. Analysis and monitoring of certain body parts, such as the heart, heart chambers, blood vessels and/or the lungs may be achieved using this impedance mapping.

A first non-limiting example takes the form of an electrical impedance tomography (EIT) device configured to be implanted within a patient, the EIT device comprising a housing, a plurality of electrodes exposed external to the housing, wherein at least a first electrode of the two or more of the plurality of electrodes is adapted for placement in an internal thoracic vein (ITV) of the patient, operational circuitry disposed within the housing, operatively coupled to the plurality of electrodes and configured to apply an impedance signal via two or more of the plurality of electrodes, and determine a set of impedances from the applied impedance signal useful to produce a tomographic image using the set of impedances, and a power source disposed within the housing and configured to power the operational circuitry.

Additionally or alternatively a second non-limiting example takes the form of an EIT device as in the first non-limiting example wherein the impedance signal is an alternating current (A/C).

Additionally or alternatively a third non-limiting example takes the form of an EIT device as in the first non-limiting example wherein the impedance signal is a direct current (D/C).

A fourth non-limiting example take the form of a system comprising an EIT device as in the first to third non-limiting examples and an external device wherein the EIT device comprises telemetry circuitry for communicating with the external device, the external device comprises telemetry circuitry for communicating with the EIT device, and the external device comprises a display operatively coupled to the operational circuitry and configured to display the tomographic image.

Additionally or alternatively a fifth non-limiting example takes the form of a system as in the fourth non-limiting example wherein the external device includes operational circuitry comprising a processor and operational instructions therefor to generate the tomographic image from data communicated by the EIT device.

Additionally or alternatively a sixth non-limiting example takes the form of a system as in the fourth non-limiting example wherein the EIT device operational circuitry is configured to generate the tomographic image and communicate the image to the external device.

Additionally or alternatively a seventh non-limiting example takes the form of an EIT as in the first to third non-limiting examples further comprising a lead adapted for placement in the ITV, operatively coupled to the operational circuitry, and including a distal portion that includes the at least one of the two or more of the plurality of electrodes, and a proximal portion having a proximal end that includes a connector for coupling to the housing.

Additionally or alternatively an eighth non-limiting example takes the form of an EIT as in the first to third non-limiting examples wherein at least a second electrode from the plurality of electrodes is adapted for placement in an intercostal vein of the patient.

Additionally or alternatively a ninth non-limiting example takes the form of an EIT as in the first to third non-limiting examples wherein at least a second electrode from the plurality of electrodes is adapted for placement in an azygous vein of the patient.

Additionally or alternatively an tenth non-limiting example takes the form of an EIT as in the first to third non-limiting examples wherein the tomographic image is used to monitor cardiac parameters.

Additionally or alternatively an eleventh non-limiting example takes the form of an EIT as in the tenth non-limiting example wherein the cardiac parameters include cardiac chamber volume for one or more cardiac chambers.

Additionally or alternatively a twelfth non-limiting example takes the form of an EIT as in the tenth non-limiting example wherein the cardiac parameters include stroke volume measurements.

Additionally or alternatively a thirteenth non-limiting example takes the form of an EIT as in the tenth non-limiting example wherein the cardiac parameters include cardiac chamber pressure.

Additionally or alternatively a fourteenth non-limiting example takes the form of an EIT as in the first to third non-limiting examples wherein the tomographic image is used to monitor respiratory parameters.

Additionally or alternatively a fifteenth non-limiting example takes the form of an EIT as in the fourteenth non-limiting example wherein the respiratory parameters include lung volumes.

Additionally or alternatively a sixteenth non-limiting example takes the form of an EIT as in the fifteenth non-limiting example wherein the lung volumes disclose asymmetric breathing.

Additionally or alternatively a seventeenth non-limiting example takes the form of an EIT as in the fourteenth non-limiting example wherein the respiratory parameters include respiratory rate.

An eighteenth non-limiting example takes the form of a method of treating a patient using at least two electrodes for use in an implantable electrical impedance tomography (EIT) system, the method comprising applying an impedance signal using the at least two electrodes, wherein at least a first electrode of the at least two electrodes is located in an internal thoracic vein (ITV) of the patient, determining a set of impedances from the applied impedance signal, and producing a tomographic image based on the set of impedances.

Additionally or alternatively a nineteenth non-limiting example takes the form of a method as in the eighteenth non-limiting example wherein the impedance signal is an alternating current (A/C).

Additionally or alternatively a twentieth non-limiting example takes the form of a method as in the eighteenth non-limiting example wherein the impedance signal is a direct current (D/C).

Additionally or alternatively a twenty-first non-limiting example takes the form of a method as in the eighteenth to twentieth non-limiting examples wherein the EIT system further includes a display and the method further comprises generating the tomographic image on the display.

Additionally or alternatively a twenty-second non-limiting example takes the form of a method as in the eighteenth to twentieth non-limiting examples wherein the EIT system further includes a lead located in the ITV and having the first electrode of the at least two electrodes disposed thereon.

Additionally or alternatively a twenty-third non-limiting example takes the form of a method as in the eighteenth to twentieth non-limiting examples wherein a second electrode from the at least two electrodes is located in an intercostal vein of the patient.

Additionally or alternatively a twenty-fourth non-limiting example takes the form of a method as in the eighteenth to twentieth non-limiting examples wherein a second electrode from the at least two electrodes is located in an azygous vein of the patient.

Additionally or alternatively a twenty-fifth non-limiting example takes the form of a method as in the eighteenth to twentieth non-limiting examples wherein the tomographic image is used to monitor cardiac parameters.

Additionally or alternatively a twenty-sixth non-limiting example takes the form of a method as in the twenty-fifth non-limiting example wherein the cardiac parameters include cardiac chamber volume for one or more cardiac chambers.

Additionally or alternatively a twenty-seventh non-limiting example takes the form of a method as in the twenty-fifth non-limiting example wherein the cardiac chamber parameters include stroke volume measurements.

Additionally or alternatively a twenty-eighth non-limiting example takes the form of a method as in the twenty-fifth non-limiting example wherein the cardiac parameters include cardiac chamber pressure.

Additionally or alternatively a twenty-ninth non-limiting example takes the form of a method as in the eighteenth to twentieth non-limiting examples wherein the tomographic image is used to monitor respiratory parameters.

Additionally or alternatively a thirtieth non-limiting example takes the form of a method as in the twenty-ninth non-limiting example wherein the respiratory parameters include lung volumes.

Additionally or alternatively a thirty-first non-limiting example takes the form of a method as in the thirtieth non-limiting example wherein the lung volumes disclose asymmetric breathing.

Additionally or alternatively a thirty-second non-limiting example takes the form of a method as in the twenty-ninth non-limiting example wherein the respiratory parameters include respiratory rate.

A thirty-third non-limiting example takes the form of a method comprising applying an impedance signal using an electrode of an implantable medical device (IMD), wherein the electrode is located in an internal thoracic vein (ITV) of the patient, determining a set of impedances from the applied impedance signal using operational circuitry of the IMD, sending the set of impedances to an external device using telemetry circuitry of the IMD, and producing a tomographic image based on the set of impedances using the external device.

Additionally or alternatively a thirty-fourth non-limiting example takes the form of a method as in the thirty-third non-limiting example wherein the impedance signal is an alternating current (A/C).

Additionally or alternatively a thirty-fifth non-limiting example takes the form of a method as in the thirty-third non-limiting example wherein the impedance signal is a direct current (D/C).

Additionally or alternatively a thirty-sixth non-limiting example takes the form of a method as in the thirty-third to thirty-fifth non-limiting examples wherein the external device includes a display and the method further comprises generating the tomographic image using on the display.

Additionally or alternatively a thirty-seventh non-limiting example takes the form of a method as in the thirty-third non-limiting example wherein the impedance signal is also applied using a second electrode of the IMD located in an intercostal vein of the patient.

Additionally or alternatively a thirty-eighth non-limiting example takes the form of a method as in the thirty-third non-limiting example wherein the impedance signal is also applied using a second electrode of the IMD located in an azygous vein of the patient.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a thoracic anatomy including the internal thoracic veins (ITVs);

FIG. 1B illustrates the posterior thoracic anatomy including placement of the azygos veins;

FIG. 2 illustrates a torso in a section view;

FIG. 3A-3B illustrates the ITV and linked vasculature in isolation;

FIG. 4 illustrates an implantable medical device (IMD);

FIG. 5 illustrates an Electrical Impedance Tomography (EIT) system;

FIG. 6A-6F illustrates thoracic anatomy with the EIT system; and

FIG. 7 is a block flow diagram for an illustrative method.

### DETAILED DESCRIPTION

The internal thoracic vein (ITV), which may also be referred to as the internal mammary vein, is a vessel that drains the chest wall and breasts. There are both left and right internal thoracic veins on either side of the sternum, beneath the ribs. The ITV arises from the superior epigastric vein, accompanies the internal thoracic artery along its course and terminates in the brachiocephalic vein. The present inventors have recognized that the ITV may make a suitable location for placement of a cardiac lead for electrical signal sensing capability to allow recognition and discrimination of atrial activity. While much of the following disclosure focuses on the use of the ITV, many of these concepts could also be applied to the internal thoracic arteries, which may sometimes be referenced as the internal mammary arteries. Some additional details related to the use of the ITV for placement of cardiac leads may be found in U.S. patent application Ser. No. 15/667,167, titled IMPLANTATION OF AN ACTIVE MEDICAL DEVICE USING THE INTERNAL THORACIC VASCULATURE, the disclosure of which is incorporated herein by reference.

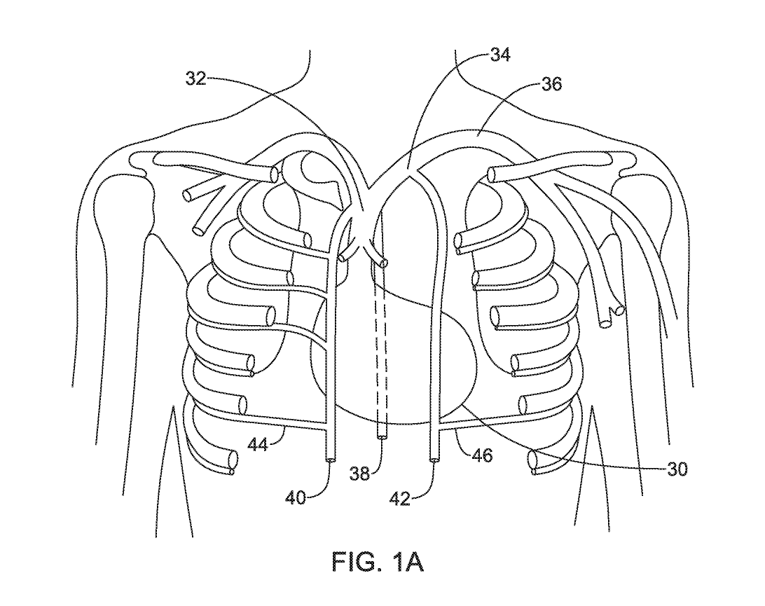

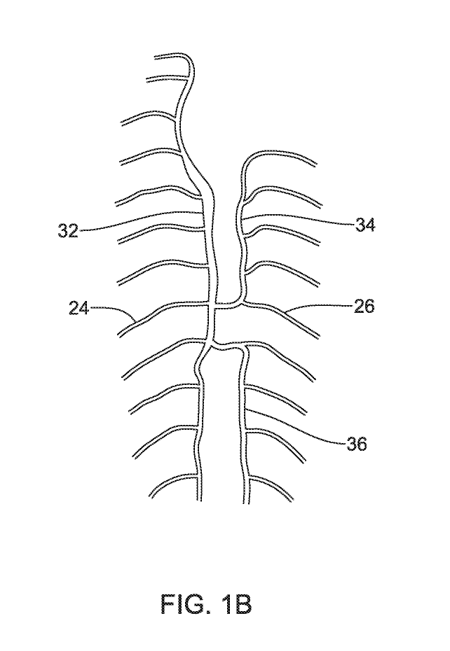

FIG. 1A illustrates the thoracic anatomy including location of the internal thoracic veins (ITVs) 40, 42. A right intercostal vein 44 may couple to the right ITV 40 and a left intercostal vein 46 may couple to the left ITV 42. The right and left intercostal veins 44, 46 may each run along a costal groove on an inferior portion of a rib. An outline of the heart is shown at 30, with the superior vena cava (SVC) shown at 32. The brachiocephalic veins 34 couple to the SVC 32 and extend past various cephalic branches to the subclavian vein 36. The azygos vein is also shown at 38.

As used herein, the “ITV” is the name applied for the vein while it runs beneath the chest, that is, superior to the lower margin of the ribs. Inferior of this location, the blood vessel is referred to (at least in this description) as the superior epigastric vein.

FIG. 1B illustrates the posterior anatomy including placement of the azygos vein, 32, accessory hemiazygos vein 34, and hemiazygos vein 36. The right intercostal vein 24 may run posteriorly along the costal groove on the inferior portion of the right rib and may couple to the azygos vein 32. The left intercostal vein 26 may run posteriorly along the costal groove on the inferior portion of the left rib and may couple to the accessory hemiazygos vein 34. In various embodiments, a left and/or right intercostal vein, at any suitable level of the torso, may be selected and used for implantation of an electrode and lead for use in delivering cardiac therapy. The intercostal veins 24, 26 may be a final implant location for a device or lead, or may provide an avenue for implantation of a device or lead in another part of the anatomy such as in the ITV, the mediastinum, and/or the azygos vein 32, hemiazygos vein 36, or accessory hemiazygos vein 34.

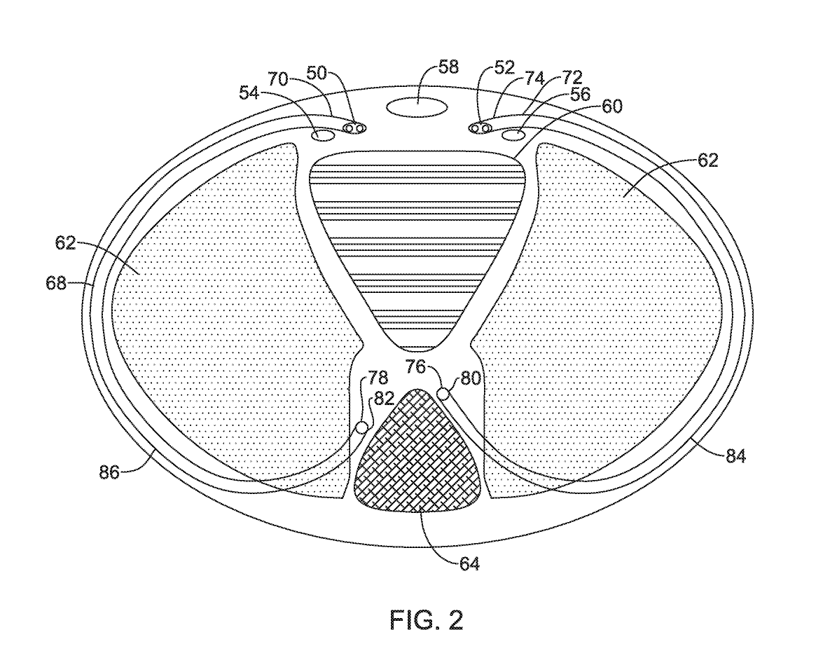

FIG. 2 shows the torso in a section view to highlight the location of various vascular structures. More particularly, in the example, the left and right ITV are shown at 50, 52, running parallel to and more central of the internal thoracic arteries 54, 56, on either side of the sternum 58. The heart is shown at 60, with the lungs at 62 and spinal column at 64. The ITV 50, 52 lie beneath the ribs but outside and separate from the pleurae of lungs 62. The ribs are omitted in the drawing in order to show the intercostal veins. The left anterior intercostal vein 68 runs along the inferior portion of a rib and couples to the left ITV 50 at junction 70, forming an ostium at the point where the left anterior intercostal vein 68 flows into the left ITV 50. Additionally, the right intercostal vein 72 runs along the inferior portion of another rib and couples to the right ITV 52 at junction 74, forming an ostium at the point where the anterior intercostal vein 72 flows into the right ITV 52.

An azygos vein and a hemiazygos vein are shown at 76, 78, respectively, running parallel to and on either side, more or less, of the spinal column 64. The azygos vein 76 and the hemiazygos vein 78 also lie beneath the ribs but outside and separate from the pleurae of lungs 62. The left posterior intercostal vein 86 couples to the hemiazygos vein 78 at a junction 82, forming an ostium at the point where the intercostal vein 86 flows into the hemiazygos vein 78. Additionally, the right posterior intercostal vein 84 couples to the azygos vein 76 at a junction 80, forming an ostium at the point where the intercostal vein 86 flows into the azygos vein 76.

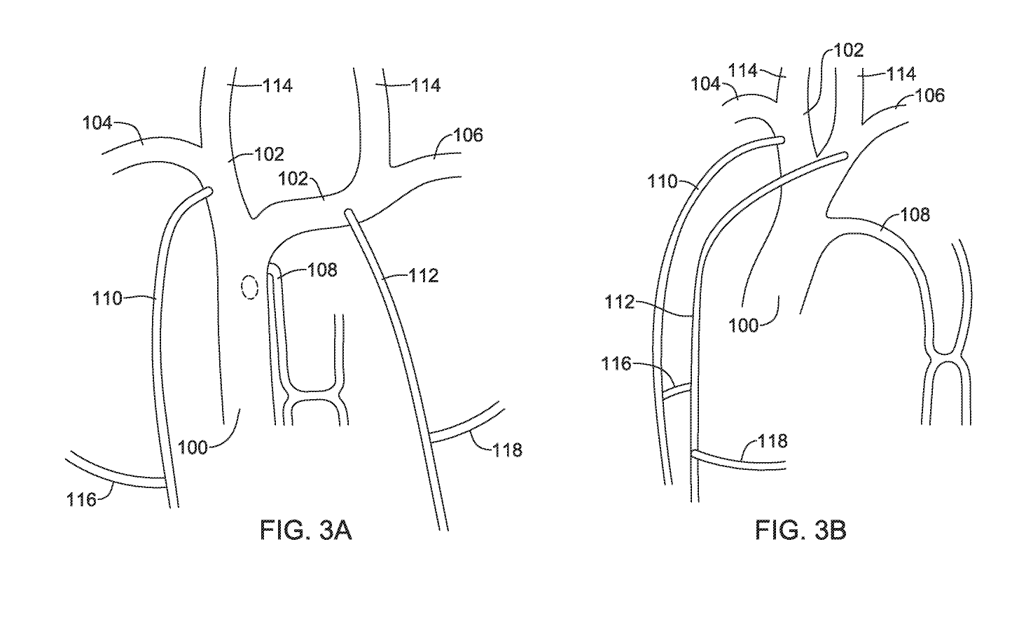

FIGS. 3A-3B show the ITV and linked vasculature in isolation. FIG. 3A is an anterior view of selected portions of the venous structure of the upper torso, and FIG. 3B is a lateral view of the same. The SVC is shown at 100, with the brachiocephalic veins 102 splitting at the upper end of the SVC. The right subclavian vein is at 104, and the left subclavian vein is at 106. The azygos vein is included in the illustration at 108, extending off the posterior of the SVC 100, and running inferiorly posterior of the heart as can be understood from the lateral view of FIG. 3B.

The right and left ITV are shown at 110, 112. These each branch off at a location that is considered part of the brachiocephalic veins 102. Selected right and left intercostal veins are shown at 116, 118. There are left and right intercostal veins along the lower margin of each of the ribs. In several embodiments the intercostal veins of the 4^{th}, 5^{th}, or 6^{th} ribs are proposed for implantation of a lead with access through the intercostal vein to the ITV. In one example, the intercostal vein of the 6^{th} rib is accessed. In other examples, access may be more superior or inferior than these locations, as desired. These may branch off at a location of the right and left ITV's and continue to run along a costal groove of an inferior portion of a the ribs. The internal jugular veins are also shown at 114.

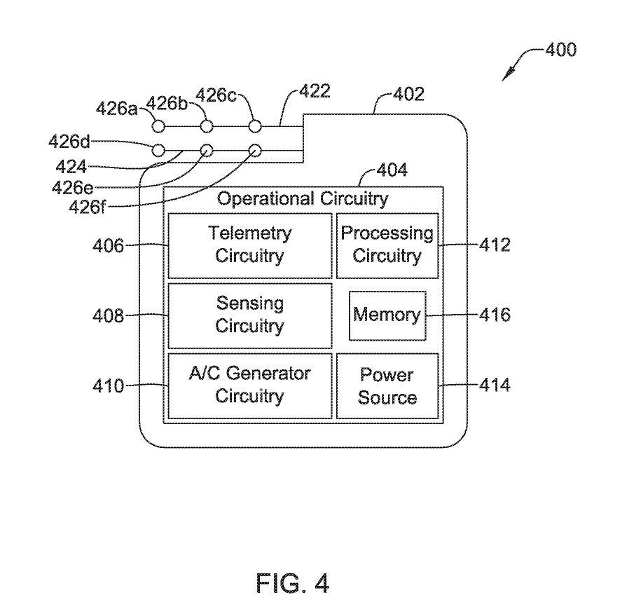

FIG. 4 depicts an illustrative implantable medical device (IMD) 400 that may be implanted into a patient and may measure parameters of body parts of the patient. For example, the IMD 400 may be an electrical impedance tomography (EIT) device. In some embodiments, the IMD 400 may also be an implantable cardioverter defibrillator (ICD). In certain embodiments, the IMD 400 may be configured to apply an impedance signal (e.g., alternating current (A/C), direct current (D/C), etc.) and determine a set of impedances from the impedance signal that may be useful to produce a tomographic image.

As can be seen in FIG. 4, the IMD 400 may have a housing 402 that encases operational circuitry 404. In certain embodiments, the housing 402 may be implanted in, for example, a thoracic region of the patient. In other embodiments, the housing may be implanted in the chest cavity, on the chest, or in an abdominal portion of the patient. The housing 402 may generally include any of a number of known materials that are safe for implantation in a human body and may, when implanted, hermetically seal the various components of the IMD 400 from fluids and tissues of the patient's body. Furthermore, in some examples, the IMD 400 may also include a header for securing one or more leads 422 and 424.

In various embodiments, the leads 422, 424 may include electrical wires that conduct electrical signals between electrodes 426A-426F and one or more circuits located within the housing 402. In some cases, the leads 422, 424 may be connected to and extend away from the housing 402 of the IMD 400. In some examples, the leads 422, 424 are implanted on, within, or adjacent to a heart of a patient or in or on the chest cavity of the patient. In some examples, the leads 422 and/or 424 may be located in an ITV, in an intercostal vein, in an azygos vein, and/or in a hemiazygos vein of a patient. One or more leads 422, 424 and/or electrodes 426A-426F may be placed subcutaneously, outside the ribs, if desired.

The leads 422, 424 may contain one or more electrodes 426A-426F positioned at various locations on the leads 422, 424, and in some cases at various distances from the housing 402. Some leads may only include a single electrode, while other leads (e.g., 422 and 424) may include multiple electrodes (e.g., 426A-426C and 426D-426F). Generally, the electrodes 426A-426F are positioned on the leads 422, 424 such that when the leads 422, 424 are implanted within the patient, one or more of the electrodes 426A-426F are positioned to perform a desired function. For example, the one or more of the electrodes 426A-426F may be positioned subcutaneously and outside of the patient's heart (e.g., in an ITV, an intercostal vein, an azygos vein, and/or a hemiazgos vein). In some cases, the electrodes 426A-426F may be implanted in the ITV, the intercostal vein, the azygos vein, and/or the hemiazgos vein such that they circumferential surround body parts of the patient in a plane and enable the IMD 400 to separate tissue components of the body parts based on differential impedances. One or more of the electrodes 426A-426F may be replaced by a different device such as an ultrasound transducer, a microphone or hydrophone, an accelerometer, an optical output an/or receiving device, a temperature sensor, or other active or passive element.

In some examples, the one or more of electrodes 426A-426F may be configured to apply an A/C that travels through a body part of the patient and is received by some or all of the remaining electrodes 426A-426F. For example, the IMD 400 may generate an A/C and the lead 422 may conduct the A/C to the electrodes 426A-426C. The electrodes 426A-426C may be located in an ITV of the patient and each electrode may apply the A/C to the chest tissue of the patient. The electrodes 426D-426F, which may be located in an intercostal vein of the patient, may each receive and/or sense the applied A/C. The lead 424 may, in turn, conduct the received A/C to the operational circuitry 404 of the IMD 400. The operational circuitry 404 may then determine a set of impedances from the received A/C that can be used to produce a tomographic image. In certain embodiments, the electrodes 426A-426F may take the form of ring electrodes, segmented electrodes, coil electrodes, or other designs. One or more transducers, such as a transducer to sense optical or mechanical (sound or motion) signals may be provided in addition to or in place of the electrodes 426A-426F. The housing 402 may serve as an electrode for sending or receiving A/C as well.

In a illustrative example, the housing 402 may be paired with electrode 426A to deliver an A/C signal to tissue of a patient. If a voltage output is delivered, then current through the electrodes delivering the signal may be used to determine impedance between that pair of electrodes. The A/C signal delivery may be repeated, choosing different pairs for A/C signal delivery until a mapping of impedance pairs is generated to provide a tomographic image. For example, the housing 402 may be paired with the other lead electrodes 426B-426F, and also electrode 426A may be paired with the other lead electrodes 426B-426F, until all pairs possible are exhausted. In an alternative, a subset of possible pairs may be used. More than two electrodes may be included, such as by delivering a signal using the housing 402 as reference, and tip electrodes 426A and 426D as the active electrodes for a sensing step. Any suitable mapping and set of combinations may be selected.

In another illustrative example, the housing 402 may be paired with electrode 426A to deliver an A/C signal to tissue of a patient. The impedance of the signal delivery pair 402/426A may be calculated if desired. In addition, the signal detected at one or more of the other electrode locations 426B-426F may also be samples/sensed, to gain an understanding of signal propagation across the various electrodes without necessarily having to test each or several individual pairs.

Different approaches to gathering impedance information from an array of electrodes may be found, for example, in U.S. Pat. No. 9,192,760, titled APPARATUS AND METHOD FOR DETERMINING THE RELATIVE POSITION AND ORIENTATION OF NEUROSTIMULATION LEADS, the disclosure of which is incorporated herein by reference. The '760 patent uses the various impedances determined during testing to identify the relative position and location of multiple leads in a patient, particularly for identifying lead migration and/or to guide neuromodulation system fitting. In the '760 patent, a direct current output is generated to gather the “real” impedance. In some embodiments of the present invention, an A/C signal is applied instead of a DC signal as in the '760 patent. In some embodiments of the present invention, a tomography mapping is generated using the impedance measurements, wherein the lead positions are known in advance and the focus is on determining features of the patient's tissue including, for example, characteristics of the heart such as cardiac chamber size and motion, and/or lung tissue characteristics.

In various embodiments, the operational circuitry 404 may include telemetry circuitry 406, sensing circuitry 408, A/C generator circuitry 410, processing circuitry 412, a power source 414, and memory 416. The IMD 400 may include more or less circuitry and modules, depending on the application. For example, A/C generator circuitry 410 may include output switches to select one or more electrodes or vectors for outputting the A/C signal, and the sensing circuitry 408 may also include input switches to select electrodes for sensing, filtering, amplification and analog-to-digital conversion circuitry to provide a signal to the processing circuitry 412. Processing circuitry 412 may use instruction sets stored in the memory 416 to perform various analyses described below and to control system operation.

In certain embodiments, the telemetry circuitry 406 may be configured to communicate with devices such as sensors, other medical devices such as a leadless cardiac pacemaker (LCP), an ICD, an implantable pulse generator (IPG), or a wearable device such as a cardiac monitor, and/or the like, that are located externally to the IMD 400. Such devices may be located either external or internal to the patient's body. Irrespective of the location, external devices (i.e. external to the IMD 400 but not necessarily external to the patient's body) can communicate with the IMD 400 via telemetry circuitry 406 to accomplish one or more desired functions. For example, the IMD 400 may communicate information, such as electrode measurements, sensed electrical signals, data, instructions, messages, etc., to an external medical device (e.g. LCP and/or a programmer or a patient's mobile device such as a phone or watch having Bluetooth or other communications capability) through the telemetry circuitry 406. The external medical device may use the communicated measurements, signals, data, instructions, messages, etc., to perform various functions, such as determining electrical conductivity of a body part, permittivity of a body part, impedance of a body part, storing received data, and/or performing any other suitable function. The IMD 400 may additionally receive information such as signals, data, instructions and/or messages from the external medical devices through the telemetry circuitry 406, and the IMD 400 may use the received signals, data, instructions and/or messages to perform various functions, such as obtaining electrode measurements, storing received data, and/or performing any other suitable function. The telemetry circuitry 406 may be configured to use one or more methods for communicating with external devices. For example, the telemetry circuitry 406 may communicate via radiofrequency (RF) signals (Bluetooth, ISM, or Medradio, for example), inductive coupling, optical signals, acoustic signals, conducted communication signals, and/or any other signals suitable for communication. Communication may also take the form of conducted communication in which electrical signals and potential differences therefrom are conducted through the body.

In the example shown in FIG. 4, the A/C generator circuitry 410 may be electrically connected to the electrodes 426A-426F. The A/C generator circuitry 410 may be configured to generate electrical stimulation signals. For example, the A/C generator circuitry 410 may generate and deliver A/C by using energy stored in the power source 414 and deliver the generated A/C via the electrodes 426A-426C and/or 426D-426F. Alternatively, or additionally, the A/C generator circuitry 410 may include one or more capacitors, and the A/C generator circuitry 410 may charge the one or more capacitors by drawing energy from the power source 414. The A/C generator circuitry 410 may then use the energy of the one or more capacitors to deliver the generated A/C via the electrodes 426A-426C and/or 426D-426F. In at least some examples, the A/C circuitry 410 may include switching circuitry to selectively connect one or more of the electrodes 426A-426F to the A/C generator circuitry 410 in order to select which of the electrodes 426A-426F (and/or other electrodes) delivers and receives the A/C. An H-Bridge is one known circuit for A/C output control. The A/C generator circuitry 410 may generate and deliver A/C to a body part with particular features or in particular sequences. The A/C generator circuitry 410 may include an oscillator circuit to allow generation of a sinusoidal output signal. In another example, the A/C circuit may include one or more digital to analog converters (DAC) to provide controlled output signals in a biphasic or other form, as desired. The A/C circuit may be configured to monitor characteristics of its output signal such as by monitoring the impedance encountered by the A/C signal such as, if a voltage controlled output is issued, by determining the current which flows. The A/C generator circuitry 410 may instead measure voltage of a current controlled output in similar manner.

The sensing circuitry 408 may be used to receive the resulting A/C in order to provide electrical signals (i.e., data) to the processing circuitry 412 indicative of the conductivity, permittivity, and/or impedance of the body part. In some cases, the A/C generator circuitry 410 may provide controllable A/C pulses. In some cases, the A/C generator circuitry 410 may allow the processing circuitry 412 to control the A/C pulse frequency, slew, width, pulse intensity, pulse shape or morphology, and/or any other suitable A/C pulse characteristic. The sensing circuitry 408 may also be controllable as by, for example, having a controllable sampling rate, frequency band, slew rate, sensitivity and/or dynamic range to accommodate different conditions in the body and/or A/C signal characteristics. The sensing circuitry 408 may include one or more analog-to-digital converter subcircuits and/or sample/hold circuitry for use in sampling the sensed signal and converting the sensed signal to a form that can be stored in memory 416 and/or processed in the processing circuitry 412. In an example, the sensing circuitry 408 may be integrated into the processing circuitry, if desired.

The processing circuitry 412 may be configured to control the operation of the IMD 400. For example, the processing circuitry 412 may be configured to receive electrical signals from the sensing circuitry 408. Based on the received signals, the processing circuitry 412 may determine, for example, conductivity, permittivity, and/or impedance of a body part. Based on any determined conductivity, permittivity, and/or impedance measurements of the body part, the processing circuitry 412 may determine and record a resulting set of equipotentials and essentially “map” the body part. The processing circuitry 412 may further control the telemetry circuitry 406 to send the equipotentials or map of the body part to an external device that may generate a tomographic image of the body part. In other examples, the processing circuitry may control the telemetry circuitry 406 to directly send the determined conductivity, permittivity, and/or impedance measurements to the external device and the external device may “map” the body part. In some examples, the processing circuitry 412 may control the telemetry circuitry 406 to receive instructions from another device (e.g., an external device). The instructions may inform the processing circuitry 412 what electrodes the A/C generator should select and activate so certain measurements may be obtain regarding a body part or a specific area of the body part.

According to various embodiments, the measurements may be obtained from the produced tomographic images. For example, tomographic images of the heart or chambers of the heart may be used to monitor parameters of the heart that may disclose the overall health or complications the heart may potentially encounter. In some cases, the monitored cardiac parameters may include, but are not limited to cardiac chamber volumes, stroke volume measurements, passive and active filling components (e.g., E & A wave surrogates), heart rate, systolic and diastolic blood pressure, mean atrial pressure, stroke volume/index, cardiac output/index, systemic vascular resistance, systemic vascular resistance index, left cardiac work, left cardiac work index, velocity index, acceleration index, heather index, PEP, LVET, thoracic fluid content, total arterial compliance, and systolic time ratio.

Similarly, tomographic images of the lungs may be used to monitor respiratory parameters of the lungs that may disclose their overall health or complications the lungs may potentially encounter. In some cases, the monitored respiratory parameters may include, but are not limited to lung volumes/lung impedance for edema, fluid accumulation, respiratory volume changes, respiratory rate, tidal volume, inspiration/expiration ratio, and airway constriction/congestion. These parameters may be indicative of certain ailments such as asymmetric breathing, asthma, wheezing, rales, etc.

In addition, the tomographic images may also be used to screen or monitor abnormalities in the body of the patient. These abnormalities may include, but are not limited to cancer for internal organs especially after treatment, valvular disease, blood clots for AF patients, dyssynchrony, pacing efficacy, abdominal aortic aneurysm, and organ internal bleeding.

In some cases, the tomographic images may also be used to monitor abdominal impedance that may be helpful in hypo- and hypertensive episodes, worsening HF (especially slow, with ascites), and right-sided heart failure. In some cases, the tomographic images may be used to monitor temporal relationships between abdominal congestion and thoracic congestion. In some cases, the tomographic images may act as a surrogate for intra-abdominal pressure or volume that may be used for diagnosis of a multitude of non-cardiac ailments.

In some examples, the processing circuitry 412 may include a pre-programmed chip, such as a very-large-scale integration (VLSI) chip and/or an application specific integrated circuit (ASIC). In such embodiments, the chip may be pre-programmed with control logic in order to control the operation of the IMD 400. For example, a state machine architecture may be used. By using a pre-programmed chip, the processing circuitry 412 may use less power than other programmable circuits (e.g. general purpose programmable microprocessors) while still being able to maintain basic functionality, thereby potentially increasing the battery life of the IMD 400.

In other examples, the processing circuitry 412 may include a programmable microprocessor. Such a programmable microprocessor may allow a user to modify the control logic of the IMD 400 even after implantation, thereby allowing for greater flexibility of the IMD 400 than when using a pre-programmed ASIC. In some examples, the processing circuitry 412 may store information on and read information from the memory 416. In other examples, the IMD 400 may include a separate memory (not shown) that is in communication with the processing circuitry 412, such that the processing circuitry 412 may read and write information to and from the separate memory.

The power source 414 may provide power to the IMD 400 for its operations. In some instances, the power source 414 may be a rechargeable battery, which may help increase the useable lifespan of the IMD 400. If the power source 414 is rechargeable, additional circuitry to receive power for recharging (such as an inductive coil) may be provided along with charging control circuits and/or safety circuitry known in the art. In still other examples, the power source 414 may be some other type of power source such as a primary cell battery, as desired. Any suitable chemistry for implantable primary cell or rechargeable batteries may be used.

The components of the operational circuitry may comprise suitable subcircuits such as ASIC or discrete chips for use in telemetry operations, digital and analog logic and/or, if desired a digital signal processor of the sensing circuitry, as well as suitable A/C frequency controlling circuitry in block 410, which may include its own frequency/oscillating circuitry or may rely upon signals from other components of the operational circuitry.

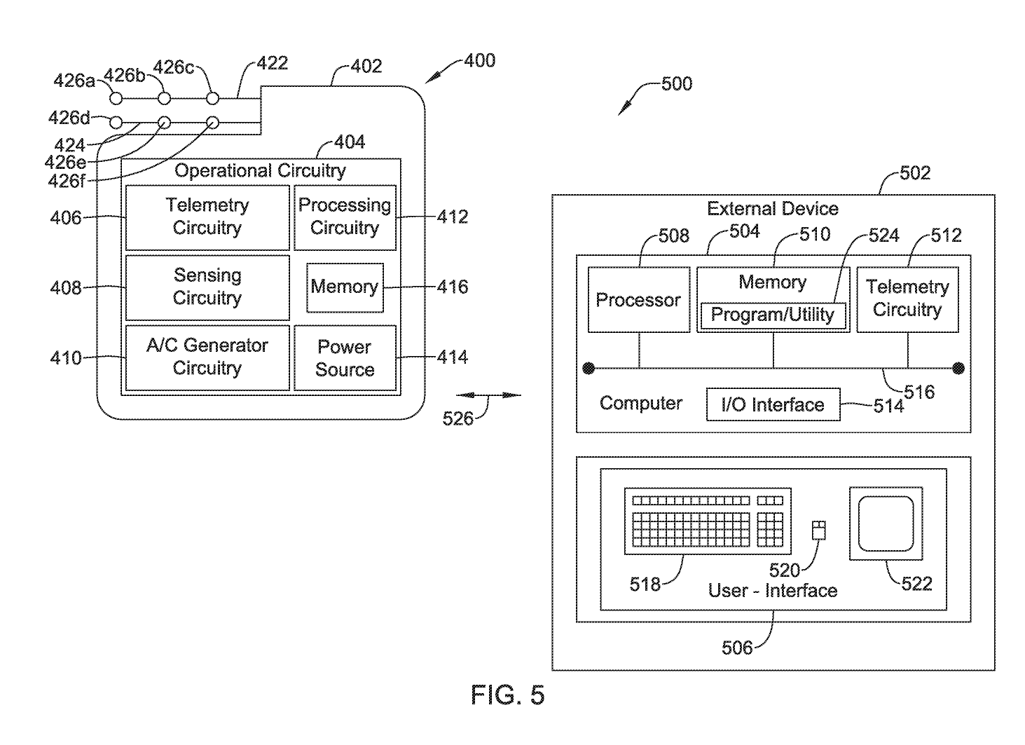

FIG. 5 depicts an exemplary system 500 comprising the IMD 400 and an external device 502 that may be utilized to perform EIT imaging. According to various embodiments, the external device 502 is of a type that is suitable for accessing and/or utilizing the IMD 400, consistent with embodiments of the present disclosure. The external device 502 provides only an illustration of one implementation and does not imply any limitations with regard to the environments in which different embodiments may be implemented. Many modifications to the external device 502 may be made based on design and implementation requirements. Examples of external devices, environments, and/or configurations that may be represented by the external device 502 include, but are not limited to, desktop computers, laptop computers, tablet computers, smartphones, server computers, thin clients, thick clients, multiprocessor systems, microprocessor-based systems, and distributed cloud computing environments. In some cases, the external device 502 merely provides a user interface for an installer or the like to interact with the IMD 400.

In certain embodiments, components of the external device 502 may include operational circuitry 504 and a user interface 506. Components of the operational circuitry 504 may include a processor 508, a memory 510, an I/O interface 514, and telemetry circuitry 512. Each of the components of the operational circuitry 504 may be connected to an internal bus 516 that includes data, address, and control buses, to allow the components of the operational circuitry 504 to communicate with each other via the bus 516.

In certain embodiments, the processor 508 may be a central processing unit (CPU) that executes an operating system and computer software executing under the operating system. In some cases, the processor 508 may also execute other instructions stored in the memory 510. The memory 510 can include computer system readable media in the form of volatile memory, such as random access memory (RAM) and/or cache memory. The memory 510 may further include other removable/non-removable, volatile/non-volatile computer system storage media. By way of example only, the memory 510 may include a storage system for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 516 by one or more data media interfaces. As will be further depicted and described below, the memory 510 may include at least one program product having a set of program modules that are configured to carry out the functions of providing instructions to the IMD 400.

In one example, program/utility 524 may be stored in the memory 510 and may include a set of application program modules (e.g. software). In some cases, the program/utility 524 may also include an operating system and program data. According to various embodiments, the application program modules may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

In various embodiments, the operational circuitry 504 may communicate with one or more external devices such as the user interface 506. In some cases, the user interface 506 may include a keyboard 518, a mouse 520, and a display 522, which enable a user to interact with the operational circuitry 504 via the I/O interface 514. A touchscreen may be provided, combining for example the keyboard 518 and display 522 together.

As stated herein, in various embodiments, the IMD 400 may communicate with one or more devices such as the external device 502. Such communication 526 can occur via the telemetry circuitry 406 of the IMD 400 and the telemetry circuitry 512 of the external device 502. The telemetry circuitry 512 may be internal to the external device 502 or may, in some examples, be provided as a wand or dongle that plugs into a port such as USB port of the external device.

For example, in some cases, the display 522 may visually display an application program from the program/utility 524 that enables a user (e.g., a physician) to create a tomographic image of a body part of a patient. The physician may then use the mouse 520 and/or the keyboard 518 to select the body part and/or region of the body part (e.g., the heart, the lungs, the right atrium of the heart, the left lung, etc.) from the application program for which to create a tomographic image. The application program instructions may then be sent to the processor 508. The processor 508 may then use the telemetry circuitry 512 to communicate 526 the instructions to the telemetry circuitry 406 of the IMD 400. The telemetry circuitry 406 may then relay the instructions to the processing circuitry 412. The instructions may inform the processing circuitry 412 what electrodes 426A-426F the A/C generator 410 should select and activate so certain measurements may be made regarding the body part or the specific region of the body part. In response, the A/C generator circuitry 410 may use the selected delivery electrodes 426A-426C to generate and deliver A/C to the body part or specific body part region and use the selected receiving electrodes 426D-426F to receive the resulting A/C. The A/C generator circuitry 410 and/or sensing circuitry 408 may then provide electrical signals (i.e., data) to the processing circuitry 412 indicative of the resulting received A/C. Based on the received electrical signals, the processing circuitry 412 may determine, for example, conductivity, permittivity, and/or impedance of the body part or body part region. Based on the determined conductivity, permittivity, and/or impedance measurements of the body part or body part region, the processing circuitry 412 may determine and record a resulting set of equipotentials and essentially “map” the body part or body part region. The processing circuitry 412 may further control the telemetry circuitry 406 to communicate 526 the map of the body part or body part region to the telemetry circuitry 512 of the external device 502. The telemetry circuitry 512 may then relay the map to the processor 508. In response, the processor 508 may generate the tomographic image of the body part or body part region and use the display 522 to display the tomographic image to the physician.

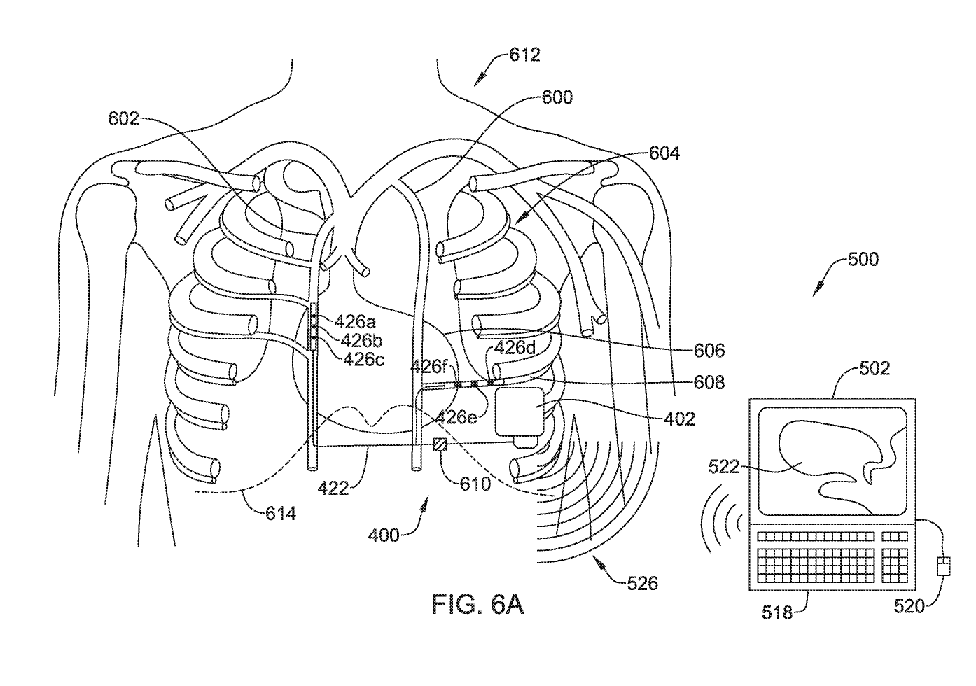

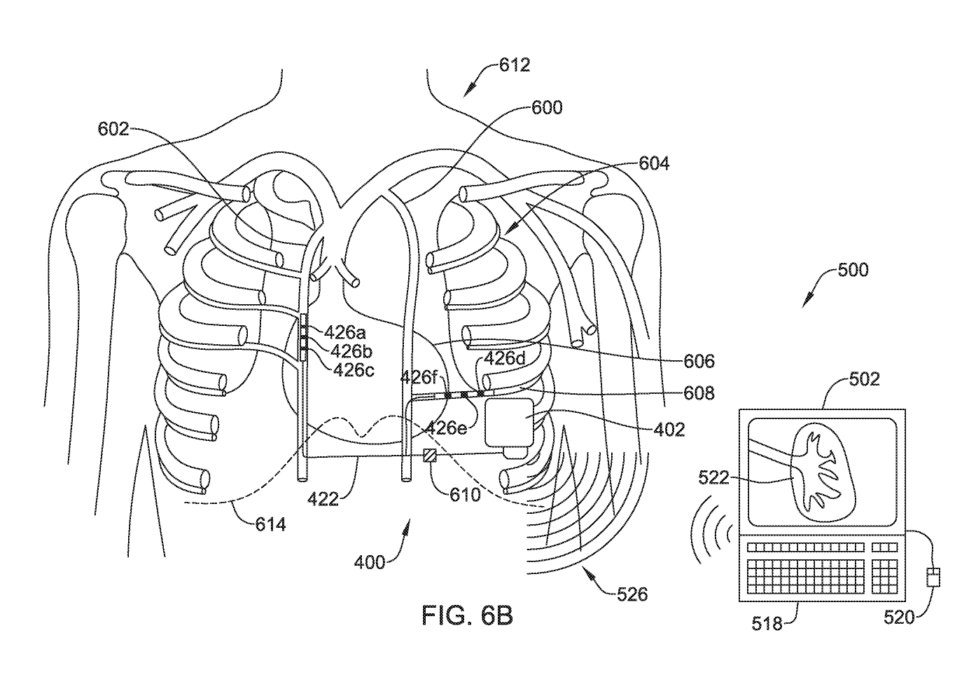

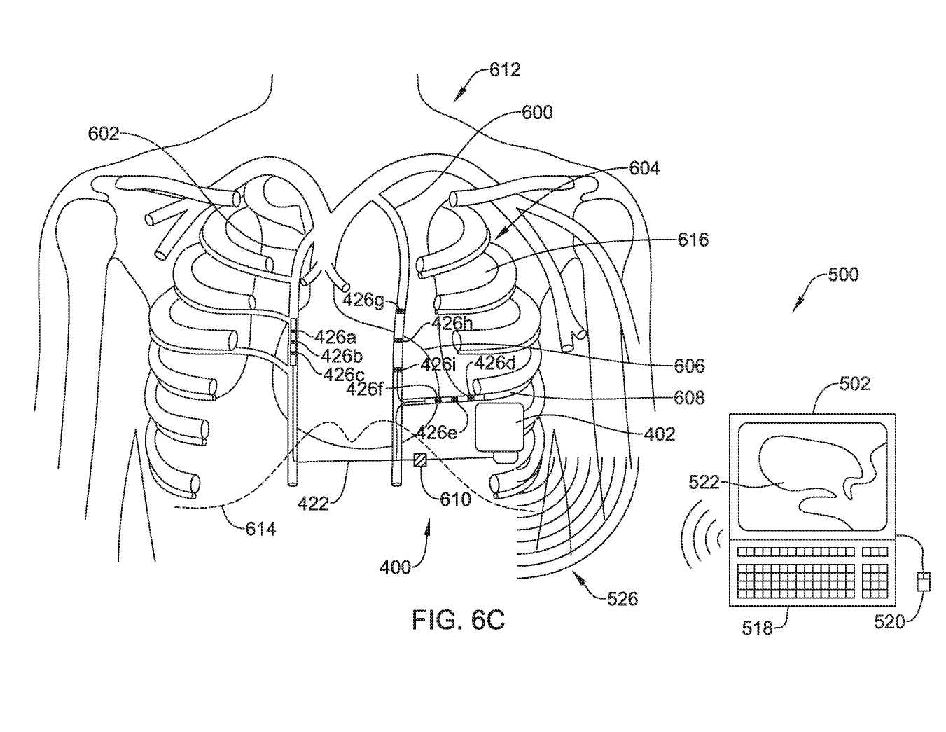

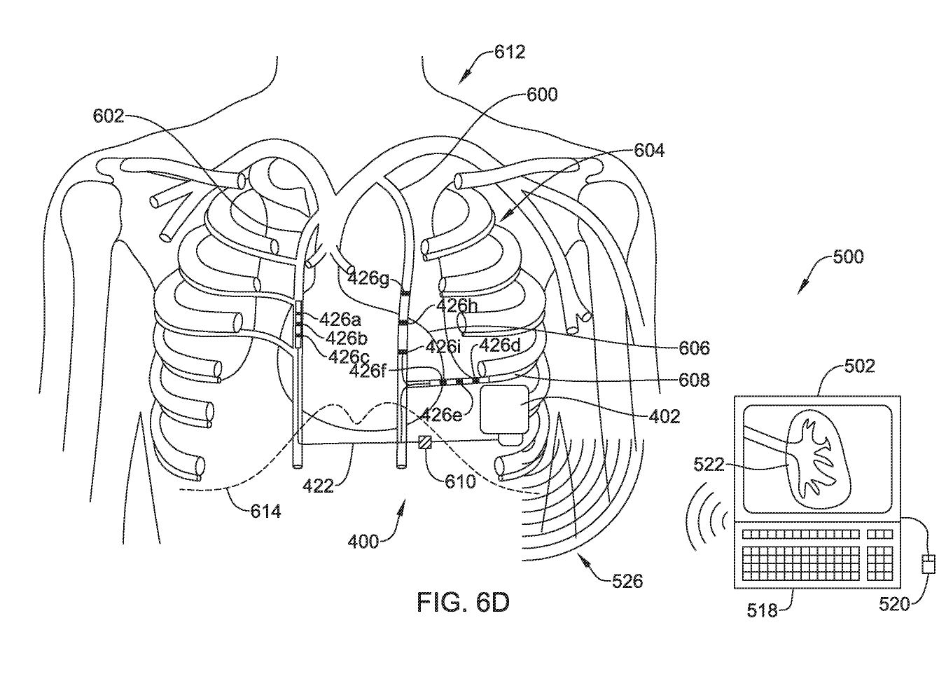

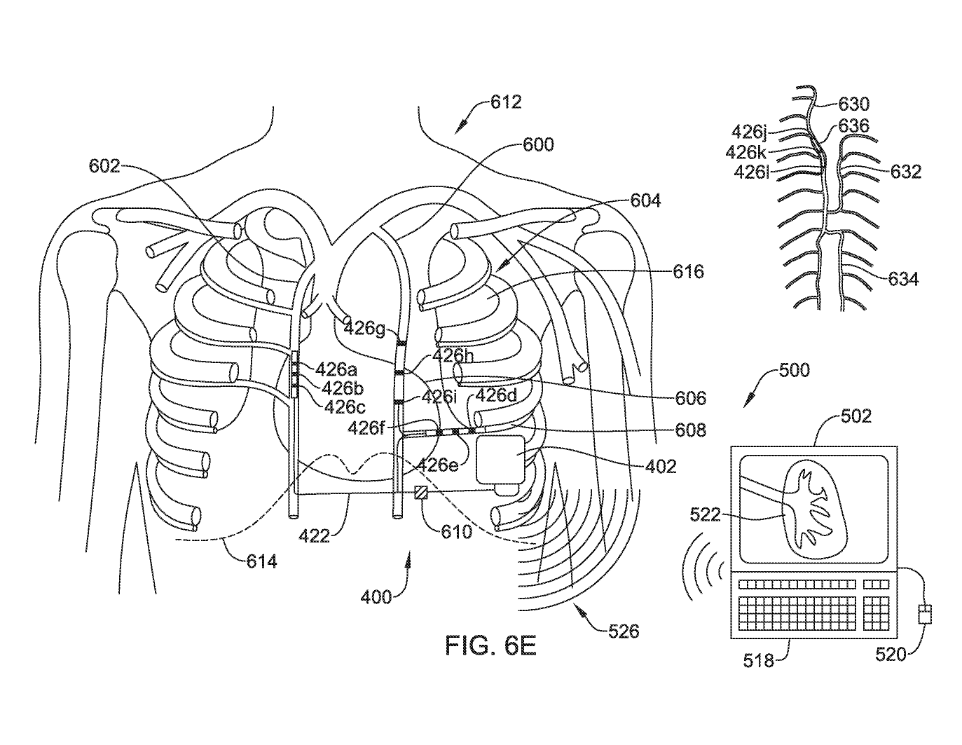

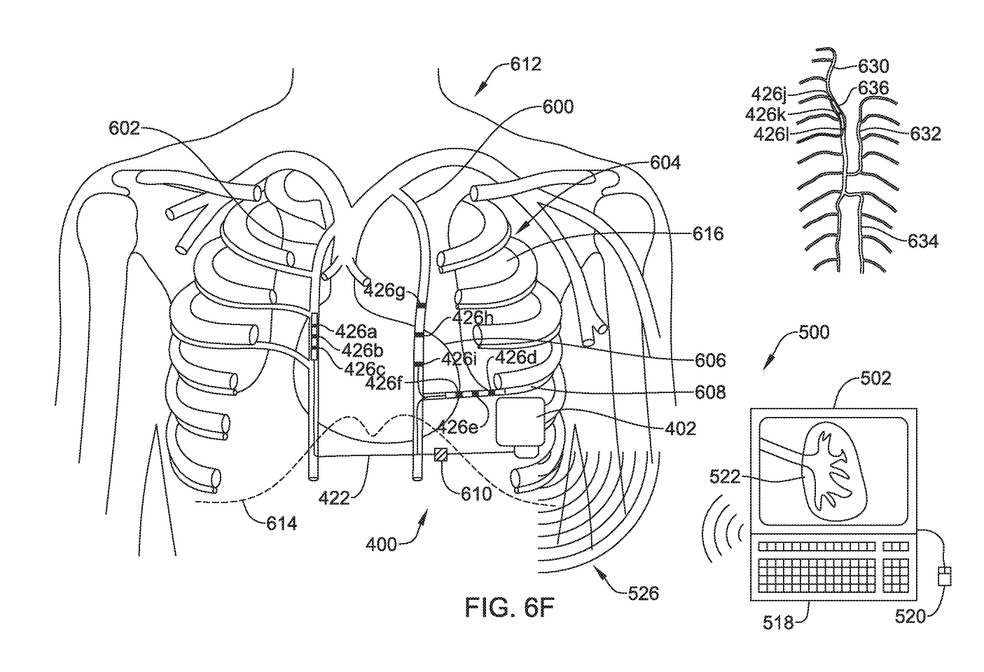

FIG. 6A depicts a first configuration of the system 500 implanted in a patient 612. FIG. 6A illustrates portions of the thoracic anatomy of the patient 612 including location of the left ITV 600 and the right ITV 602. The ribcage is shown at 604 and an outline of the heart is shown at 606. The system 500 is also shown having the IMD 400 (e.g., an EIT device) with the lead 422 located in the right ITV 602 and the lead 424 in an intercostal vein 608 of the patient. In certain embodiments, as shown, the lead 422 may include the electrodes 426A-426C spaced from one another and the lead 424 may include the electrodes 426D-426F spaced from one another. According to various embodiments, the distal portion of the leads 422 and 424 may include a fixation apparatus or shape for the flexible lead, such as a 2 or 3 dimensional curve, tines, an expandable member, hooks, a side-extending engagement structure, etc. A number of examples for lead shape and design for implantation using the ITV may be found in U.S. patent application Ser. No. 15/846,060, titled LEAD WITH INTEGRATED ELECTRODES, U.S. Provisional Patent Application No. 62/452,537, titled IMPLANTABLE MEDICAL DEVICE, and U.S. Provisional Patent Application No. 62/486,635, titled ACTIVE MEDICAL DEVICE WITH ATTACHMENT FEATURES, the disclosures of which are incorporated herein by reference.

Access to the ITVs 600 and 602 may be achieved at any location, such as superior or inferior positions. FIG. 6A shows implantation from an inferior position in the right ITV 602 and in a left intercostal vein 608. In this example, the right ITV 602 and left ITV 600 have been accessed by introduction through their respective superior epigastric veins from a location inferior to the rib margin 614. In an alternative approach the musculophrenic vein may be used instead of the superior epigastric vein. The housing 402 of the IMD 400 has been placed at approximately the left axilla. The housing 402 may be placed as desired, for example at the anterior axillary line, the midaxillary line, or in the posterior axillary line.

In the illustration, a suture sleeve is shown at 610 and is used to fixate the leads 422, 424, for example, to the subcutaneous fascia. For placement of the lead 422, the right ITV 602 may be accessed and a tunnel established between the left axilla and the access location such as along a portion of the inframammary crease. The lead 422 may, in this case, be relatively stiff to assist in keeping it emplaced in the patient as shown, if desired. The tunnel may also be used for placement of the lead 424 in the intercostal vein 608.

In the example of FIG. 6A, a left axillary housing location is shown; a right sided, pectoral or subclavicular left or right position may be used instead, in combination with the right ITV 602 placement, the left ITV 600 placement, intercostal vein placement, azygos vein placement, and/or hemiazygos vein placement. Illustrative examples of additional implant locations are shown in U.S. patent application Ser. No. 15/846,081, titled IMPLANTATION OF AN ACTIVE MEDICAL DEVICE USING THE INTERCOSTAL VEIN and U.S. patent application Ser. No. 15/868,799, titled IMPLANTATION OF AN ACTIVE MEDICAL DEVICE USING THE INTERNAL THORACIC VASCULATURE, the disclosures of which are incorporated herein by reference.

The ITV's 600, 602 may be accessed via their corresponding superior epigastric veins using standard access techniques known in the art for implanting traditional transvenous pacemakers and defibrillators. For example, access may be achieved using ultrasound guided needle insertion. The access method may resemble the Seldinger technique. Other venipuncture or cutdown techniques may be used instead.

The Seldinger technique may include creating a puncture at the desired access location, with a hollow needle or trocar, for example under ultrasound guidance, introducing a guidewire through the needle and into the desired blood vessel, removing the needle, keeping the guidewire in place, and then inserting an introducer sheath, which may have a valve at its proximal end, over the guidewire. The introducer sheath may be advanced to a location to place its distal tip near a desired location. Contrast injection may be useful to visualize the superior epigastric vein, ITV and/or intercostal vein structures. A guide catheter and guidewire may then be introduced through the introducer sheath. The guidewire may be the same as used in gaining initial access (if one is used to gain access), or may be a different guidewire. In another example, a cutdown technique may be used to access the desired superior epigastric vein by incision through the skin. The incision may be made laterally from the location of the desired vein. Next, possibly after visual confirmation the desired vessel is accessed, incision into the selected vein can be made, followed by insertion of the lead. Once access to the right superior epigastric vein is achieved, the vessel can be traversed in a superior direction to place the lead 422 with the electrodes 426A-426C at the desired level by entering the right ITV. Similarly, once access to the left superior epigastric vein is achieved, the vessel can be traversed in the superior direction into the left ITV 600. The lead 424 with electrodes 426D-426F may then enter an ostium from the left ITV 600 into the intercostal vein 608 and be place at a desired location in the intercostal vein 608.

Various approaches for use of the ITV are shown in U.S. Provisional patent application Ser. No. 15/801,719, titled PARASTERNAL PLACEMENT OF AN ACTIVE MEDICAL DEVICE USING THE INTERNAL THORACIC VASCULATURE, U.S. patent application Ser. No. 15/814,990, titled TRANSVENOUS MEDIASTINUM ACCESS FOR THE PLACEMENT OF CARDIAC PACING AND DEFIBRILLATION ELECTRODES, and U.S. Provisional Patent Application No. 62/473,882, titled IMPLANTABLE MEDICAL DEVICE, the disclosures of which are herein incorporated by reference.

The leads 422, 424 may be tunneled from the parasternal access location across and down to the housing 402, which may be implanted at the left axilla as illustrated. For ease of illustration the housing 402 is shown at about the anterior axillary line, level with the cardiac apex and/or inframammary crease. In other examples the housing 402 may be more lateral and/or posterior, such as at the mid-axillary line or posterior axillary line, or may even be more dorsal with placement dorsally between the anterior surface of the serratus and the posterior surface of the latissimus dorsi. A right sided axillary, pectoral or subclavicular left or right position may be used instead, in combination with right, left ITV, intercostal vein, or azygos vein placement.

In some examples, a flexible lead may be introduced with the support of a guide catheter during advancement. The guide catheter may receive the lead through a guide catheter lumen that serves to retain a fixation apparatus or shape for the flexible lead, such as a 2-dimensional or 3-dimensional curvature, tines, an expandable member, or hooks or a side-extending engagement structure. A stylet may be placed through the lead, or a portion thereof, to retain a straight shape during implantation; upon removal of the stylet, a curvature may then be released for securing the lead in place.

In another alternative, the guide catheter and guidewire may be omitted by providing a lead with a flexible or steerable structure, and/or a lead configured for implantation using a steerable stylet. For example, a lead may be configured to be implanted using a steerable stylet in a lumen thereof, with the initial placement into the left ITV 600 (or right ITV 602, or an intercostal vein, or an azygos vein if desired) at the distal end of the introducer sheath, possibly using contrast visualization, if desired. Once initial access is achieved, simply pushing the stylet should be sufficient to implant the lead to a desired location in the ITV, intercostal vein, and/or azygos vein. The stylet may have a secondary function of preventing an anchoring structure of the lead from assuming an anchoring shape or releasing an anchoring tine, hook, expandable member, stent or other device. In other examples, a guidewire and/or sheath may not be needed. Due to the limited angulation required for accessing the ITV from a parasternal incision, the lead may be inserted directly into the ITV, reducing the time and complexity of the procedure.

The leads 422, 424 shown in FIG. 6A include the three ring electrodes 426A-426C and 426D-426F. The ring electrodes 426A-426C and 426D-426F may serve as A/C delivering and A/C receiving electrodes. The housing 402 may also include an electrode or electrodes for delivering and receiving A/C.

FIG. 6A also depicts the system 500 in which the IMD 400 may perform EIT mapping and communicate the mapping to the external device 502 for producing a tomographic image. According to various embodiments, an application program may be displayed on the display 522 of the external device 522. A user (e.g., a physician) may create a tomographic image of a body part region of the patient by using the mouse 520 and/or the keyboard 518 to select the body part region from the application program. In this example, the user has chosen to create a tomographic image of the right atrium of the heart 606. In other examples, another body part or body part region may be selected, such as the left atrium of the heart, the right ventricle of the heart, the left ventricle of the hearth, a lung, or a specific region of a lung. Once the user has submitted their request to the application program, the external device 502 may communicate 526 instructions to the IMD 400 to perform EIT.

In certain embodiments, the IMD 400 may be an absolute EIT device. An absolute EIT device may be used to assist in the digital reconstruction of static images or two-dimensional representations of a body part of the patient 612. In some cases, an absolute EIT device may apply an A/C that travels three-dimensionally along the path of least resistivity. Accordingly, because muscle and blood conduct A/C better than fat or bone, the absolute EIT device allows for the construction of static images of the body part.

In another embodiment, the IMD 400 may be a time difference EIT device. A time difference EIT device may record measurements of a body part between two or more physiological states associated with linear conductivity changes. An example of this approach is heart tissue during pumping due to linear conductivity changes between intake and expulsion which are caused by varying contents of conductive blood during each pump cycle. This permits digital subtraction of recorded measurements obtained during the pump cycle and results in functional images of heart operation. One major advantage is that relative changes of conductivity remain comparable between measurements even if one of the recording electrodes is less conductive than the others, thereby reducing most artifacts and image distortion. Similarly, another example of this approach is lung tissue during breathing due to linear conductivity changes between inspiration and expiration which are caused by varying contents of insulating air during each breath cycle. This permits digital subtraction of recorded measurements obtained during the breath cycle and results in functional images of lung ventilation.

Based on the instructions from the external device 502, the IMD 400 may select which electrodes 426A-426F to apply and receive A/C. That is, the electrodes used to deliver the A/C signal and/or used to measure propagation of the A/C signal may be selected in view of the anatomical structure selected by the physician. In certain embodiments, the leads 422 and 424 may be implanted in the right ITV 602 and the intercostal vein 608 such that the electrodes 426A-426F at least partly circumferentially surround the right atrium of the heart 606 and enable the IMD 400 to separate tissue components of the right atrium based on differential impedances.

In certain embodiments, to avoid muscle or cardiac stimulation (e.g., stimulation of the myocardium), a stimulation threshold may be determined. In certain embodiments, the threshold may be determined by applying pulses at rates higher than the intrinsic heart rate. The IMD 400 may then observe the paced rate frequencies that match the stimulating pulse frequencies. In other embodiments, a strength-duration curve may be implemented to arrive at appropriate excitation amplitudes. In these embodiments, at high frequencies (e.g., smaller PW), a higher threshold may exist and allow for larger excitation signals, such as between 10-100 kHz. In addition, the electrode configuration, spacing, and surface area may affect the actual strength-duration curve. Accordingly, the stimulation threshold may also be affected. In yet further embodiments, the intrinsic pulse may be sensed and A/C signals may be sent during cardiac refractory.

In this example, the stimulation threshold may be determined to be greater than 100 kHz. As such, the electrodes 426A and 426B may be selected to apply the A/C at a few milli-amperes and at a frequency of 10-100 kHz, to the right atrium of the heart 606. In some cases, the A/C may be applied at a single frequency. In other cases, multiple frequencies may be applied to potentially better differentiate between the heart 606 tissue of the right atrium. In some cases, the electrodes 426D and 426E may be selected to receive the resulting A/C.

In various embodiments, the A/C applied is relatively small and below the threshold at which they may cause significant muscle and/or nerve stimulation. Furthermore, the frequency of the A/C may be sufficiently high as to not give rise to electrolytic effects in the patient 612 and the Ohmic power dissipated may be sufficiently small and diffused over the body of the patient 612 to be easily handled by the body's thermoregulatory system. These properties may allow the A/C from the electrodes 426A and 426B to be continuously applied in the patient 612, such as for example, during mechanical ventilation in an intensive care unit (ICU). In some cases, the IMD 400 may be used for continuous real time visualization of operation of right atrium. Moreover, resolution may be improved by increasing the number of electrodes disposed on the leads 422 and 424. Image quality may be further improved by constructing the system 500 to include active surface electrodes external to the patient 612, which may reduce signal loss, artifacts, and interferences associated with cables as well as cable length and handling.

In certain embodiments, once the resulting A/C is received the IMD 400 and sensing circuitry 408 thereof may use an analog demodulation circuit to convert the A/C to a direct current level before running it through an analog to digital converter. In other embodiments, the IMD 400 may convert the A/C directly before performing digital demodulation. The IMD 400 may then make conductivity, permittivity, and/or impedance measurements of the right atrium. In some cases, if the applied A/C had multiple frequencies, the IMD 400 may also be capable of measuring both magnitude and phase of the received A/C. The IMD 400 may then use the measurements to determine and record a resulting set of equipotentials and map the right atrium. The IMD 400 may then communicate 526 the map data to the external device 502 and the external device 502 may generate a tomographic image. In other embodiments, the IMD 400 may communicate the determined conductivity, permittivity, and/or impedance measurements to the external device and the external device may map the right atrium and generate a tomographic image.

According to various embodiments, if the tomographic image is to be displayed in real-time, the external device 502 may include an application that utilizes regularized inverse of a linearization of the forward problem or a fast version of a direct reconstruction method such as the D-bar method. In some cases, the external device 502 may generate a ‘difference image’, that is, differences in voltage between two time points are left-multiplied by the regularized inverse to calculate an approximate difference between permittivity and conductivity images. In another embodiment, the external device 502 may construct a finite element model of the right atrium and adjust the conductivities, for example, using a variant of Levenburg-Marquart method to fit the measured data. These are just a few examples of how the external device 502 may generate the tomographic image of the right atrium from the map data. In any event, once the tomographic image has been generated, the external device 502 may display the tomographic image on the display 522.

In other embodiments, the IMD 400 may use the map data and generate the tomographic image of the right atrium. In these cases, the IMD 400 may use similar methodologies as the external device 502 to produce the tomographic image. The IMD 400 may then communicate 526 the image data to the external device 502 and the external device 502 may display the tomographic image of the right atrium on the display 522.

In various embodiments, the tomographic image of the right atrium may be used to monitor cardiac parameters that indicate the overall health of the heart 606 and/or complications the heart 606 may potentially encounter. In addition, the system 500 may obtain tomographic images of other chambers of the heart 606, the whole heart 606, and/or areas related to the heart 606 that may be used to monitor the cardiac parameters. As stated herein, these cardiac parameters may include, but are not limited to cardiac chamber volumes, stroke volume measurements, passive and active filling components (e.g., E & A wave surrogates), heart rate, systolic and diastolic BP, mean aterial pressure, stroke volume/index, cardiac output/index, systemic vascular resistance, systemic vascular resistance index, left cardiac work, left cardiac work index, velocity index, acceleration index, heather index, PEP, LVET, thoracic fluid content, total arterial compliance, and systolic time ratio. Moreover, the EIT images may also be used to screen or monitor abnormalities of the heart. These abnormalities may include, but are not limited to cancer, valvular disease, blood clots for AF patients, dyssynchrony, pacing efficacy, and abdominal aortic aneurysm.

FIG. 6B also depicts the first configuration of the system 500 implanted in the patient 612, similar to FIG. 6A. According to various embodiments, the system 500 may operate similar to the operation described in regard to FIG. 6A, however, in this example the IMD 400 may perform EIT mapping of a left lung 616 of the patient 612 and communicate the mapping to the external device 502 for producing a tomographic image. According to various embodiments, the application program may once again be displayed on the display 522 of the external device 522. This time the physician may create a tomographic image of the left lung 616 by using the mouse 520 and/or the keyboard 518 to select the left lung 616 from the application program. Once the user has submitted their request to the application program, the external device 502 may communicate 526 instructions to the IMD 400 to perform EIT.

Since images of a larger area must be produced this time, the instructions may tell the IMD 400 to select all of the electrodes 426A-426C on the lead 422 to apply the A/C and all the electrodes 426D-426F on the lead 424 to receive the resulting A/C. The IMD 400 may then make conductivity, permittivity, and/or impedance measurements of the left lung 616. The IMD 400 may then use the measurements to determine and record a resulting set of equipotentials and map the left lung 616. The IMD 400 may then communicate 526 the map data to the external device 502 and the external device 502 may generate a tomographic image. Once the tomographic image has been generated, the external device 502 may display the tomographic image on the display 522. In other embodiments, the IMD 400 may communicate the determined conductivity, permittivity, and/or impedance measurements to the external device 502 and the external device 502 may map the left lung 616 and generate a tomographic image. In still further embodiments, the IMD 400 may use the map data and generate the tomographic image of the left lung 616. The IMD 400 may then communicate 526 the image data to the external device 502 and the external device 502 may display the tomographic image of the left lung 616 on the display 522.

In various embodiments, the EIT images of the left lung 616 may be used to monitor respiratory parameters that may disclose overall health or complications the lungs may potentially encounter. As stated herein, these respiratory parameters may include, but are not limited to lung volumes/lung impedance for edema, fluid accumulation, respiratory volume changes, respiratory rate, tidal volume, inspiration/expiration ratio, and airway constriction/congestion. These parameters may be indicative of ailments such as asymmetric breathing, asthma, wheezing, rales, etc.

FIG. 6C depicts a second configuration of the system 500 implanted in the patient 612. In this configuration the lead 422 is located in the right ITV 602 and the lead 424 is located in the intercostal vein 608. In addition, the IMD 400 has a third lead 628 with electrodes 426G-426I disposed thereon and located in the left ITV 600 of the patient. In this example, the IMD 400 once again performs EIT mapping of the right atrium of the heart 606 and communicates the mapping to the external device 502 for producing a tomographic image. According to various embodiments, the system 500 may operate similar to the operation described in regard to FIG. 6A, however, in this example the resolution of the tomographic image of the right atrium may be improved because there are more electrodes to circumferential surround the right atrium.

FIG. 6D also depicts the second configuration of the system 500 implanted in the patient 612, similar to FIG. 6C. According to various embodiments, the system 500 may operate similar to the operation described in regard to FIG. 6C, however, in this example the IMD 400 may perform EIT mapping of the left lung 616 of the patient 612 and communicate the mapping to the external device 502 for producing a tomographic image. In certain embodiments, the resolution of the tomographic image of the left lung 616 may be improved from the example described in regard to FIG. 6B because there are more electrodes to circumferential surround the left lung 616.

FIG. 6E depicts a third configuration of the system 500 implanted in the patient 612. FIG. 6E illustrates portions of the thoracic anatomy of the patient 612 including location of the left ITV 600 and the right ITV 602, similar to FIGS. 6A-6D. In addition, FIG. 6E illustrates the posterior anatomy including placement of the azygos vein, 630, accessory hemiazygos vein 632, and hemiazygos vein 634. In this configuration the lead 422 is located in the right ITV 602, the lead 424 is located in the intercostal vein 608, and the lead 628 is located in the left ITV 600. In addition, the IMD 400 has a fourth lead 636 with electrodes 426J-426L disposed thereon and located in the azygos vein 630. For example, lead 636 may wrap around the patients right side by passing through an intercostal vein to the posterior of the patient to reach the azygos vein 630. In other examples, a lead may extend around the left side of the patient to one of the hemiazygos or accessory hemiazygos veins 634, 632.

In this example, the IMD 400 once again performs EIT mapping of the right atrium of the heart 606 and communicates the mapping to the external device 502 for producing a tomographic image. According to various embodiments, the system 500 may operate similar to the operation described in regard to FIG. 6A, however, in this example the resolution of the tomographic image of the right atrium may once again be improved because there are more electrodes to circumferential surround the right atrium.

FIG. 6F also depicts the third configuration of the system 500 implanted in the patient 612, similar to FIG. 6E. According to various embodiments, the system 500 may operate similar to the operation described in regard to FIG. 6E, however, in this example the IMD 400 may perform EIT mapping of the left lung 616 of the patient 612 and communicate the mapping to the external device 502 for producing a tomographic image. In certain embodiments, the resolution of the tomographic image of the left lung 616 may once again be improved from the example described in regard to FIG. 6D because there are more electrodes to circumferential surround the right atrium.

The configurations depicted in FIGS. 6A-6F provide only illustrations of three implementation configurations and do not imply any limitations with regard to configurations in which different embodiments may be implemented. Many modifications to the configurations may be made based on design and implementation requirements. Specifically, implantation configurations of electrodes in the ITV's, the intercostal veins, the azygos veins, and the hemiazygos veins are all considered and alternate configurations may be dependent upon providing the optimum circumferential electrodes in a plane configuration for the EIT imaging of a specific body part.

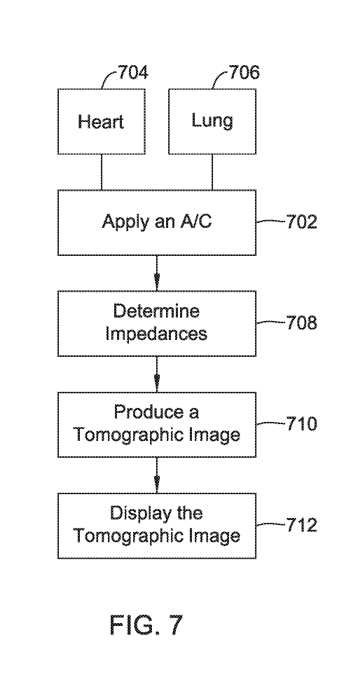

FIG. 7 is a block flow diagram for an illustrative method of treating a patient using an electrical impedance tomography (EIT) system. In some embodiments, the method may be used to create a tomographic image of a body part of the patient. As shown at 700, the method comprises applying an impedance signal 702, determining impedances 708, producing a tomographic image 710, and displaying the tomographic image 712.

For example, in some embodiments, the EIT system may include an IMD and an external device and the IMD may include at least two electrodes. Furthermore, applying the impedance signal 702 may include delivering the an A/C 704 to the body part (e.g., the heart 706, a lung 708, etc.) from one of the electrodes located in an ITV of the patient and receiving the resulting A/C using the other electrode located in an intercostal vein, an azygos vein, and/or a hemiazygos vein of the patient.

In an example, determining impedances 710 may include converting the resulting impedance signal to a direct current level before running it through an analog to digital converter. In other embodiments, the impedance signal may be converted directly before performing digital demodulation. Impedance measurements may then be made and sent to the external device. In an example, producing the tomographic image 712 may include the external device utilizing an algorithm or application program that takes the impedance data and generates a tomographic image based on the impedance data. In an example, the EIT system may further include a display and the tomographic image may then be displayed 714 on the display.

In another example, once the impedances 710 are determined, the IMD may then use the measurements to determine and record a resulting set of equipotentials and map the body part. The map data may then be sent to the external device that produces the tomographic image 712 by utilizing an algorithm or application program that takes the map data and generates a tomographic image based on the map data. The tomographic image may then be displayed 714 on the display.

In yet another example, the IMD may produce the tomographic image 712 by utilizing an algorithm or application program that takes the map data and generates a tomographic image based on the map data. The tomographic image data may then be sent to the external device and the tomographic image may be displayed 714 on the display.

Any suitable designs and materials may be used for the leads and electrodes shown and described above. Implantation tools and components may also be of any suitable material and design to allow implantation of the leads and devices described above to the locations discussed.

The implantable systems shown above may include an implantable pulse generator (IPG) adapted for use in a cardiac therapy system. The IPG may include a hermetically sealed canister that houses the operational circuitry of the system. The operational circuitry may include various elements such as a battery, and one or more of low-power and high-power circuitry. Low-power circuitry may be used for sensing cardiac signals including filtering, amplifying and digitizing sensed data. Low-power circuitry may also be used for certain cardiac therapy outputs such as pacing output, as well as an annunciator, such as a beeper or buzzer, telemetry circuitry for RF, conducted or inductive communication (or, alternatively, infrared, sonic and/or cellular) for use with a non-implanted programmer or communicator. The operational circuitry may also comprise memory and logic circuitry that will typically couple with one another via a control module which may include a controller or processor. High power circuitry such as high power capacitors, a charger, and an output circuit such as an H-bridge having high power switches may also be provided for delivering, for example, defibrillation therapy. Other circuitry and actuators may be included such as an accelerometer or thermistor to detected changes in patient position or temperature for various purposes, output actuators for delivering a therapeutic substance such as a drug, insulin or insulin replacement, for example.

Some illustrative examples for hardware, leads and the like for implantable defibrillators may be found in commercially available systems such as the Boston Scientific Teligen™ ICD and Emblem S-ICD™ System, Medtronic Concerto™ and Virtuoso™ systems, and St. Jude Medical Promote™ RF and Current™ RF systems, as well as the leads provided for use with such systems.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An electrical impedance tomography (EIT) device configured to be implanted within a patient, the EIT device comprising:
a housing;
a plurality of electrodes exposed external to the housing, wherein at least a first electrode of the plurality of electrodes is adapted for placement in an internal thoracic vein (ITV) of the patient;
operational circuitry disposed within the housing, operatively coupled to the plurality of electrodes and configured to apply an impedance signal via two or more of the plurality of electrodes, and determine a set of impedances from the applied impedance signal useful to produce a tomographic image using the set of impedances; and
a power source disposed within the housing and configured to power the operational circuitry.

2. A system comprising the EIT device of claim 1 and an external device; wherein:
the EIT device comprises telemetry circuitry for communicating with the external device;
the external device comprises telemetry circuitry for communicating with the EIT device; and
the external device comprises a display operatively coupled to the operational circuitry and configured to display the tomographic image.

3. The system of claim 2 wherein the external device includes operational circuitry comprising a processor and operational instructions therefor to generate the tomographic image from data communicated by the EIT device.

4. The system of claim 2 wherein the EIT device operational circuitry is configured to generate the tomographic image and communicate the image to the external device.

5. The EIT device of claim 1, further comprising a lead adapted for placement in the ITV, operatively coupled to the operational circuitry, the lead including a distal portion having one or more of the plurality of electrodes; and a proximal portion having a proximal end that includes a connector for coupling to the housing.

6. The EIT device of claim 1 wherein at least a second electrode from the plurality of electrodes is adapted for placement in an intercostal vein of the patient.

7. The EIT device of claim 1 wherein at least a second electrode from the plurality of electrodes is adapted for placement in an azygous vein of the patient.

8. The EIT device of claim 1 wherein the tomographic image is used to monitor cardiac chamber volume for one or more cardiac chambers.

9. The EIT device of claim 1 wherein the tomographic image is used to monitor cardiac stroke volume.

10. The EIT device of claim 1 wherein the tomographic image is used to monitor cardiac chamber pressure.

11. The EIT device of claim 1 wherein the tomographic image is used to monitor lung volume.

12. The EIT device of claim 1 wherein the tomographic image is used to monitor for asymmetric breathing.

13. The EIT device of claim 1 wherein the tomographic image is used to monitor respiratory rate.

14. A method of treating a patient using at least two electrodes for use in an implantable electrical impedance tomography (EIT) system, the method comprising:
applying an impedance signal using the at least two electrodes, wherein at least a first electrode of the at least two electrodes is located in an internal thoracic vein (ITV) of the patient;
determining a set of impedances from the applied impedance signal;
producing a tomographic image based on the set of impedances; and, either:
communicating the tomographic image or data related thereto to a second device; or
displaying the tomographic image.

15. The method of claim 14 wherein the EIT system further includes a lead located in the ITV and having at least the first electrode of the at least two electrodes disposed thereon.

16. The method of claim 14 wherein a second electrode from the at least two electrodes is located in an intercostal vein of the patient.

17. The method of claim 14 wherein a second electrode from the at least two electrodes is located in an azygous vein of the patient.

18. The method of claim 14 further comprising using the tomographic image to monitor cardiac parameters.

19. The method of claim 14 further comprising using the tomographic image to monitor respiratory parameters.

20. A method comprising:
applying an impedance signal to the upper thorax of a patient using an electrode of an implantable medical device (IMD), wherein the electrode is located in an internal thoracic vein (ITV) of the patient;
determining a set of impedances from the applied impedance signal using operational circuitry of the IMD;
sending the set of impedances to an external device using telemetry circuitry of the IMD; and
producing a tomographic image based on the set of impedances using the external device.
